(19) United States
(12) Patent Application Publication
(10) Pub. No.: US 2003/0217374 A1
(43) Pub. Date: Nov. 20, 2003

(54) Cloning B and T lymphocytes

(75) Inventor: Michael D. West, Southborough, MA (US)

Correspondence Address: CROWELL & MORING, L.L.P., Intellectual Property Group, P.O. Box 14300, Washington, DC, 20044-4300, US

(73) Assignee: Advanced Cell Technology

(21) Appl. No.: 10/342,336

(22) Filed: Jan. 15, 2003

Related U.S. Application Data
(60) Provisional application No. 60/348,130, filed on Jan. 15, 2002.

(51) Int. Cl. A01K 67/027

(52) U.S. Cl. 800/6; 800/14; 800/15; 800/16; 800/17; 800/18; 800/19

(57) ABSTRACT

This invention includes methods for producing non-human mammals expressing monoclonal or oligoclonal B or T lymphocytes, as well as embryonic and hematopoietic stem cells that differentiate into monoclonal or oligoclonal B or T cells, using cloning by nuclear transfer with a B or T cell of interest as the nuclear donor cell.

### CROSS-REFERENCE TO RELATED APPLICATIONS

This application claims priority to U.S. Provisional No. 60/348,130 filed Jan. 15, 2002, which is incorporated by reference in its entirety herein.

### FIELD OF INVENTION

The present invention concerns the production of animals having monoclonal or oligoclonal T and/or B cells via nuclear transfer, and the use of such animals to produce cells and antibodies for therapy of cancer and viral diseases. Such animals are also useful as experimental models, i.e. for studying mechanisms of allelic exclusion and the effects of somatic rearrangements, particularly as they pertain to immunoglobulin and T cell receptor diversity.

### BACKGROUND OF THE INVENTION

The past decade has been characterized by significant advances in the science of cloning, and has witnessed the birth of cloned sheep, i.e. “Dolly” (Roslin Bio-Med), goats (Genzyme Transgenics), cattle (Advanced Cell Technology), mice (WO 0145500) and pigs (PPL Therapeutics Incorporated). More recently, Advanced Cell Technology reported the isolation of the first cloned human pre-embryo produced by nuclear transfer from adult cells. Cibelli et al., 2001, e-biomed: J. Regenerative Med. 25-32. It is now clear that nuclear transfer may be performed using the nucleus from an adult, differentiated cell, which undergoes “reprogramming” when it is introduced into an enucleated oocyte. See U.S. Pat. No. 5,945,577, herein incorporated by reference in its entirety. Embryonic stem-like cells may also be isolated from the inner cell mass (ICM) cells of such a nuclear transfer unit, and differentiated in vitro into virtually any cell type of the body. See U.S. Pat. No. 6,235,970, herein incorporated reference.

The fact that embryos and embryonic stem cells may be generated using the nucleus from an adult differentiated cell has exciting implications for the fields of organ, cell and tissue transplantation. There are currently thousands of patients waiting for a suitable organ donor, who face problems of both availability and incompatibility in their wait for a transplant. If embryonic stem cells generated from the nucleus of a cell taken from a patient in need of a transplant could be made and induced to differentiate into the cell type required in the transplant, then the problem of transplantation rejection and the dangers of immunosuppressive drugs could be precluded. This technology is even more promising when considered with recent advances in tissue engineering, opening up the possibility of creating entire tissues and organs from cloned cells. Such methodology is discussed in copending U.S. Ser. No. 09/655,815, which is herein incorporated by reference.

As discussed in U.S. Pat. No. 6,235,970, embryonic stem cells or cells isolated from the inner cell mass of nuclear transfer units (cultured ICM cells or CICM cells) may be induced to differentiate into a desired cell type according to known methods. For example, as disclosed therein, embryonic stem cells may be induced to differentiate into hematopoietic stem cells, muscle cells, cardiac muscle cells, liver cells, cartilage cells, epithelial cells, urinary tract cells, etc., by culturing such cells in differentiation medium and under conditions which provide for cell differentiation. Medium and methods that result in the differentiation of CICM cells are known in the art, as are suitable culturing conditions.

For example, Palacios and colleagues teach the production of hematopoietic stem cells from an embryonic cell line by subjecting cells to an induction procedure comprising initially culturing aggregates of such cells in a suspension culture medium lacking retinoic acid followed by culturing in the same medium containing retinoic acid, followed by transferal of cell aggregates to a substrate which provides for cell attachment. Palacios et al, 1995, Proc. Natl. Acad. Sci. USA, 92:7530-7537. Others have shown that the in vitro derivation of hematopoietic cells from mouse ES cells is enhanced by addition of stem cell factor (SCF), IL-3, IL-6, IL-11, GM, CSF, EPO, M-CSF, G-CSF, LIF. Keller et al, 1993, Mol. Cell Biol. 13:473486; Kennedy et al, 1997, Nature 386(6624): 488-493,1997; Biesecker et al, 1993, Exp. Hematology, 21:774778. Murine ES cells can also generate hematopoietic stem cells (thyl⁺, SCA-I⁺, c-kit receptor⁺, lineage restricted marker negative (B-220, Mac-1, TEN 119, JORO 75. for B-lymphocyte, myeloid, erythroid, T-lymphocyte, respectively)) when cultured on a stromal cell line in the presence of IL-3, IL-6 and fetal liver stromal cell line cultured supernatant. See U.S. Pat. No. 6,245,566, herein incorporated by reference.

Moreover, Pedersen, J. Reprod. Fertil. Dev., 6:543-552 (1994) reviews numerous articles disclosing methods for in vitro differentiation of embryonic stem cells to produce various differentiated cell types including hematopoietic cells, muscle, cardiac muscle, nerve cells, among others. Further, Bain et al, Dev. Biol., 168:342-357 (1995) teaches in vitro differentiation of embryonic stem cells to produce neural cells that possess neuronal properties. Benveniste et al, Cell. Immunol., 127(1): 92-104 (1990) teaches in vitro directed differentiation of bone marrow precursors down a T cell lineage in medium supplemented with supernatant from a thymoma cell line.

More recently, researchers from the University of South Florida demonstrated that it is possible to induce human or mouse bone marrow stromal cells (BMSC), which normally give rise to bone, cartilage, and mesenchymal cells, to differentiate into neuron-like cells by culturing them in the presence of rat fetal mesencephalic or striatal cells. See Sanches-Ramos et al (August 2000) Exp. Neurol. 164(2): 247-56. Accordingly, it may be possible to mimic the environmental signals that induce pluripotent cells to differentiate along a given pathway in vitro merely by exposing pluripotent cells to differentiated cells.

Thus, using known methods and culture medium, one skilled in the art may culture CICM cells and other pluripotent cells to obtain desired differentiated cell types, e.g., neural cells, muscle cells, hematopoietic cells, etc. Therapeutic uses of such differentiated cells, particularly human cells, are unparalleled. For example, as discussed in U.S. Pat. No. 6,235,970, diseases and conditions treatable by such “isogenic” cell therapy include, by way of example, spinal cord injuries, multiple sclerosis, muscular dystrophy, diabetes, liver diseases, i.e., hypercholesterolemia, heart diseases, cartilage replacement, burns, foot ulcers, gastrointestinal diseases, vascular diseases, kidney disease, urinary tract disease, neural diseases such as Parkinson's disease, and aging related diseases. In particular, human hematopoietic stem cells may be used in medical treatments requiring bone marrow transplantation. Such procedures are used to treat many diseases, e.g., late stage cancers such as ovarian cancer and leukemia, as well as diseases that compromise the immune system, such as AIDS. Indeed, U.S. Pat. No. 6,235,970 contemplates producing human hematopoietic stem cells following nuclear transfer of an adult somatic cell from a cancer or AIDS patient, e.g., an epithelial cell or a lymphocyte, and the use of such stem cells in the treatment of diseases including cancer and AIDS.

Despite the realization that any differentiated cell may be used as a donor for nuclear transfer, and that cells and tissues derived therefrom have utility in transplantation therapies in general, the use of donor cells having particular genetic attributes, e.g. chromosomal rearrangements accumulated by way of the differentiation process, has not been described. Further, potential applications relating to the use of specially differentiated donor cells, such as B cells expressing a particular immunoglobulin from recombined heavy and light chain genes or T cells expressing a particular T cell receptor from recombined alpha and beta genes, have not been realized. Thus, the art is lacking in instruction as to how to best utilize specially differentiated cells, such as B cells and T cells, as donors for nuclear transfer, and how animals and cells generated therefrom could be used for therapeutic purposes.

### SUMMARY OF THE INVENTION

The present invention concerns methods of nuclear transfer using donor cells that contain chromosomal rearrangements, and the use of animals and cells obtained therefrom for therapeutic and experimental purposes. In particular, the invention encompasses methods for producing non-human mammals having monoclonal or oligoclonal peripheral B cell and T cell repertoires.

For instance, one embodiment includes the steps (a) identifying and isolating a non-human mature B cell of interest or a nucleus from a non-human mature B cell of interest, (b) introducing the mature B cell or its nucleus or its chromosomes into a non-human enucleated oocyte or other suitable recipient cell to form a nuclear transfer (NT) unit, (c) implanting the NT unit into the uterus of a suitable surrogate mother, and (d) permitting the NT unit to develop into a non-human mammal having a monoclonal or oligoclonal B cell repertoire.

The invention also encompasses methods for producing non-human animals and preferably mammals having monoclonal or oligoclonal T cell receptor repertoires, for instance, by (a) identifying and isolating a non-human CD4+ or CD8+ T cell of interest or the nucleus from such a T cell (b) introducing the T cell of interest or its nucleus or its chromosomes into a non-human enucleated oocyte or other suitable recipient cell to form a nuclear transfer (NT) unit; (c) implanting the NT unit into the uterus of a suitable surrogate mother; and (d) permitting the NT unit to develop into a non-human animal having a monoclonal or oligoclonal T cell receptor repertoire. Non-human animals with a monoclonal or oligoclonal peripheral B cell and/or T cell repertoires produced by the disclosed methods are also encompassed, as are the nuclear transfer units, embryos and fetuses.

The invention also includes various methods for isolating isogenic hematopoietic stem cells having the identical genotype as a given donor T cell or B cell. Such hematopoietic stem cells may be isolated directly from cloned animals in the case of non-human donors, i.e., by further including the step of (e) isolating fetal liver hematopoietic stem cells (HSCs) from said non-human fetus, wherein said HSCs differentiate into B cells that express the desired immunoglobulin or T cells that express a T cell receptor of interest. Cells isolated from cloned animals and preferably mammals by the disclosed in vitro differentiation methods, including hematopoietic stem cells, CD4+ and CD8+ T cells, B cells, and any other immune cell or immune cell precursor stem cell are also encompassed in the invention.

Hematopoietic stem cells, as well as cloned B cells and T cells, may also be isolated following in vitro differentiation of embryonic stem-like cells obtained from the inner cell mass (ICM) of a nuclear transfer unit, for example, by the culture of such ICM-derived cells that have or have not been passaged into ES lines in the presence of fetal liver endothelial cells. Such directed differentiation has particular use in producing human pluripotent and hematopoietic stem cells via nuclear transfer. For instance, one such method comprises the steps (a) identifying and isolating a human or non-human mature B cell or T cell or the nucleus therefrom, (b) introducing the mature B or T cell or its nucleus or its chromosomes into a human or non-human enucleated oocyte or other suitable recipient cell, i.e., embryonic or hematopoietic stem cell, to form a nuclear transfer (NT) unit, (c) activating the resultant NT unit, (d) culturing said activated NT unit until at least a size suitable for obtaining cultured totipotent stem cells (for example, inner cell mass (ICM) cells), (e) disassociating said activated NT unit to obtain isolated ICM cells, (f) culturing said ICM cells obtained from said cultured NT unit to obtain embryonic stem cells, and (g) permitting or directing said stem cells to develop into hematopoietic stem cells (HSCs) and isolating said HSCs, wherein said HSCs differentiate into B cells that express the desired immunoglobulin or T cells that express a T cell receptor of interest. Alternatively, the ICM cells may be directly differentiated without first making ES lines or isolating embryonic stem cells.

Particular embodiments whereby donor cells are genetically modified to prevent immunoglobulin and/or T cell receptor diversification in cloned animals are also included. For instance, a donor B cell of interest may be genetically altered with at least one insertion, deletion or disruption that would inhibit the rearrangement of Ig genes, and specifically V(H) gene replacement, in peripheral B cells of cloned animals. Likewise, a donor T cell of interest may be genetically altered with at least one insertion, deletion or disruption that would inhibit receptor revision of T cell receptor beta genes. Particularly preferred donor cells contain genetic alterations that inhibit expression of rag1 and rag2, such as homozygous deletions or inhibition of expression via antisense, RNA interference, or some other form of transcriptional or translational regulation.

The present invention further includes methods for producing in large scale oligoclonal antibodies or a monoclonal antibody of interest using the animals produced by the disclosed methods. For instance, large scale isolation of a desired monoclonal antibody may comprise immunizing a non-human cloned mammal created by the methods described herein with an antigen recognized by the immunoglobulin expressed by the original B cell of interest, and isolating the antibody of interest from the peripheral blood of the cloned mammal. Alternatively, pre-B cells or mature B cells may be isolated from such a non-human cloned mammal, and used to produce specific antibody either by in vitro immunization or other activation, i.e. LPS stimulation. Such methods find particular utility in producing human immunoglobulins, wherein the original donor B cell of interest is a mouse cell that produces a human immunoglobulin (i.e., B cells from a Xenomouse, or atransgenic mouse expressing a rearranged human Ig). Immunoglobulins produced by the disclosed methods are also included in the invention.

The present invention further includes methods of therapy using cells and/or antibodies isolated from the animals or embryonic stem cells or ICM cells disclosed herein, particularly for the treatment of cancer or viral diseases. In cancer therapeutic methods, for instance, the mature donor B cell of interest used in the disclosed nuclear transfer methods produces an immunoglobulin that binds with specificity to a tumor antigen, e.g., EGF receptor. Alternatively, donor T cells producing a desirable T cell receptor (TcR) may be used, e.g., a TcR that binds specifically to a cancer associated molecule, either alone or displayed in the context of MHC.

In methods for treating viral diseases, mature donor B cells of interest may be used that produce immunoglobulin that binds with specificity to a viral antigen. Alternatively, donor T cells producing a desirable T cell receptor may be used, e.g., a TcR that binds specifically to a virally expressed protein, either alone or displayed in the context of MHC. Particularly useful donor T cells express TcR specific for AIDS-infected cells, in that such donor T cells may be used to produce isogenic hematopoietic stem cells that may be used to reconstitute hematopoietic populations in AIDS patients, for instance patients whose CD8 cells have become defective.

### DETAILED DESCRIPTION OF THE INVENTION

B and T lymphocytes derive from hematopoietic stem cells by a series of separate differentiation events. The key events in mature B cell development occur in the fetal liver and, in adult mammals, in the bone marrow, and involve intermediate cell types designated pro- and pre-B cells. Development centers around the assembly of genetic elements encoding the immunoglobulin (Ig) cell surface receptor, which is a heterodimeric molecule consisting of heavy (H) and light (L) chains, both of which have regions that contribute to the binding of antigen and are highly variable from one Ig molecule to another. See Paul's Fundamental Immunology, 3^{rd} ed., 1993, Raven Press, N.Y., herein incorporated by reference.

The genetic elements encoding the variable regions of the Ig heavy or light chain—deemed V, D and J elements—are not contiguous on the chromosome. Rather, a series of genetic rearrangements occurs in both the heavy and light chain genes during the development of pro- and pre-B cells resulting in the construction of an expressible gene. For instance, B cell precursors rearrange the Ig heavy chain locus following an ordered sequence of events in which a D segment joins to a J segment on both chromosomes, followed by a variable (V) gene joining to the D-J_{H} segment. Once an in-frame V(D)J rearrangement results, the protein is thought to be expressed on the cell surface associated with light chain, and may deliver a signal to the cell to stop further rearrangement in the heavy chain locus thereby resulting in allelic exclusion and the expression of a single Ig molecule. Nourrit et al., 1998, J. Immunol. 160:4254-4261. Although, it was recently proposed that allelic exclusion may be the result of progression to a developmental stage that precludes rearrangement at the other Ig(H) allele, rather than a direct signal per se. Chang et al., 1999, J. Exp. Med. 189(8): 1295-1305.

T lymphocytes also derive from hematopoietic tissue, generally in the thymus. Although, several cites for extrathymic T cell maturation have also been proposed, including the bone marrow, mesenteric lymph nodes and the gut. See Dejbakhsh-Jones and Strober, 1999, Immunol. 96(25): 14493-98. Mature T cells are divided into two distinct classes depending on the cell-surface receptor they express. The majority of T cells express heterodimeric T cell receptors (TcR) consisting of α and β chains, however, a small group of T cells express receptors made up of γ and δ chains.

Among the α/β T cells, two sub-lineages are recognized as those that express the CD4 coreceptor and those that express CD8. These cells differ fundamentally in how they recognize antigen and mediate different regulatory and effector functions. For instance, CD4+ T cells are the main regulatory cells of the immune system, and may further differentiate upon stimulation into T helper (T_{H1}) cells that mainly produce IL2, interferon-γ and lymphotoxin and are effective inducers of cellular immune responses, or TH2 cells that mainly produce IL4, IL5, IL6 and IL10 and are effective to stimulate B cells to develop into antibody producing cells. See Paul's Fundamental Immunology, id. CD8+ cells, on the other hand, can develop into cytotoxic T lymphocytes (CTLs) capable of efficiently lysing target cells that express antigens recognized the particular CTL. Paul, id.

The T cell receptor of the T cell differs from the immunoglobulin of the B cell in the way it recognizes its target antigen. While the B cell receptor may bind to individual antigenic epitopes on soluble molecules or surfaces, the T cell receptor recognizes antigen in association with a major histocompatibility complex MHC molecule on the surface of an “antigen-presenting” cell (APC). The T cell receptor is similar to the Ig receptor, however, in that both receptors consist of two chains that undergo somatic rearrangement during the process of maturation, thereby resulting in receptor diversity.

Similar recombinatorial mechanisms as used in B cell Ig gene expression are used to assemble the V regions of the TcR chains. For instance, the TcR beta chain also consists of V, D and J regions that are separate in the germ line but joined together as the T cell matures. Assembly of TcR α/β genes begins upon recombination activating gene (RAG) expression and TcR β recombination in CD4(−)CD8(−)CD25(+) thymocytes. TcR β expression leads to clonal expansion, RAG down-regulation and TcR β allelic exclusion. At the subsequent CD4(+)CD8(+) stage, RAG gene expression is reinduced and V(D)J recombination is initiated at the TCR a locus. This second wave of RAG expression is terminated upon expression of a positively selected α/β TcR. See Yannoutsos et al. 2001, J. Exp. Med. 194(4): 471-80. Finally, double positive CD4(+)CD8(+) cells further differentiate into mature thymocytes that express either CD4 or CD8 and high levels of TcR in the context of a TcR-CD3 complex.

One recent reference reports that immature thymocytes have the intrinsic ability to rearrange and express two different TCR V α chains on the cell surface, and that a CD45-dependent positive selection signal mediates allelic exclusion, expression of the activated TcR V α chain, and down-regulation of the second non-selected TCR V α chain, thereby leading to mature thymocytes and peripheral T cells that only express one TCR V α chain on the cell surface. See Boyd et al., 1998, J. Immunol. 161: 1718-27. Another group recently reported that post-translational mechanisms regulating assembly of the heterodimers on the cell surface also contribute to allelic exclusion of the TcR. Sant' Angelo et al., 2001, Proc. Natl. Acad. Sci. USA 98(12): 6824-29.

Thus, although developing T and B lymphocytes have the potential to rearrange two TcR or Ig alleles, most T and B cells express only one receptor on the cell surface due to allelic exclusion. Recent evidence suggests, however, that allelic exclusion may not provide a fail-safe end to receptor diversification. Some of the first clues came with the production of transgenic animals expressing rearranged immunoglobulin genes specific for a particular antigen. For instance, Lo and colleagues reported in 1991 that expression of a transgene mouse Ig in mice and pigs did not suppress expression of endogenous IgM. Lo et al., 1991, Eur. J. Immunol. 21(4): 1001-6. Likewise, Costa and colleagues reported in 1992 that “leakiness” was consistently observed in transgenic mice expressing rearranged Ig heavy chain transgenes. Costa et al., 1992, Proc. Natl. Acad. Sci. USA 89: 2205-08. In 1996, Cascalho and colleagues reported the development of a quasi-monoclonal mouse containing one rearranged V(D)J segment at the heavy chain locus specific for the hapten (4-hydroxy-3-nitrophenyl) acetyl (NP), with the other heavy chain allele being non-functional. Cascalho et al., 1996, Science 272(5268): 1585. While the primary repertoire of this mouse was monospecific, somatic hypermutation and secondary rearrangements were shown to change the specificity of 20% of the antigen receptors on B cells.

Subsequently, Cascalho et al introduced homozygous, nonfunctional RAG-2 alleles into the quasi-monoclonal (QM) mouse and found the secondary repertoire was no longer diversified. See Cascalho et al., 1999, Dev. Immunol. 7(1): 43-50. Another group has also shown that simultaneous introduction of mu heavy chain and lambda light chain transgenes into RAG2(−/−) mice leads to the generation of a substantial population of monoclonal peripheral B cells that are functional with regard to Ig secretion. See Young et al., 1994, Genes Dev. 8(9): 1043-57. These experiments led the Cascalho group to conclude that the secondary V-gene replacement in the QM mouse is mediated by RAG-driven V(D)J recombination and not by other recombination systems. Dev. Immunol., 1999, id.

Still others have shown that secondary V(H) gene replacement may change the original heavy chain gene rearrangement having a first antigen specificity into a recombined gene having a new antigen specificity, and that such an event is selective rather than instructive because V(H) gene replacement intermediates were detected before and after immunization. See Madan et al., Eur. J. Immunol. 2000, 30(8): 2404-11. Such secondary rearrangements accompanied by hypermutation were shown to generate sufficient B cell diversity in QM mice to mount protective antiviral antibody responses via new antibody specificities. See Lopez-Macias et al., 1999, J. Exp. Med. 189(11): 1791-98. Thus, it is now thought that V(H) replacement in Ig heavy chains may play a role in the normal diversification of the antibody repertoire, particularly in later stages of development occurring in secondary lymphoid tissues. Bertrand et al., 1998, Eur. J. Immunol. 28(10): 3362-70.

Similar receptor editing mechanisms are also operative on the TcR. For instance, McMahan and Fink recently reported the age-dependent accumulation of Vβ5(−) CD4(+) T cells in Vβ5 transgenic mice, whereby endogenous V elements were expressed via a CD28-dependent process. See McMahan and Fink, 2000, J. Immunol. 165(12): 6902-7. Given that the revised repertoire was surprisingly diverse and that the recreation of the non-transgenic repertoire was dependent on CD28 expression, McMahan and Fink concluded that receptor revision occurs extrathymically. Further, the authors concluded that T cell receptor revision probably contributes to the flexibility of the immune repertoire and may even play a role in the maintenance of peripheral T cell tolerance. See MaMahan and Fink, 1998, Immunity 9(5): 637-47. Another group has recently reported the expression of RAG genes in peripheral CD4+ T cells undergoing secondary rearrangements, suggesting that RAG-mediated recombination also plays a role in receptor revision of the TcR as it does the Ig heavy chain in B cells. See Lantelme et al., 2000, J. Immunol. 164:3455-59.

The existence of receptor revision of the TcR in mature T cells is consistent with previous reports of leakiness in allelic exclusion in transgenic mice. For instance, Listman and colleagues reported in 1996 that TcR β chain transgenic mice expressing the V β 8.2 transgene still responded to all antigenic stimuli tested despite the showing that over 98% of T cells in the mice expressed the transgene. See Listman et al., 1996, Cell. Immunol. 167(1): 4455. However, it is also possible that allelic exclusion may be overcome by the functional rearrangement of both TcR β loci. Indeed, studies on human and mouse lymphocytes have shown that 1% of peripheral T cells have two functional TcR β chains expressed on the cell surface. Kersh et al., 1998, J. Immunol. 161: 585-593. Allelic exclusion and receptor revision were two phenomena taken into account in the present invention.

The present invention concerns methods of nuclear transfer using donor cells that contain chromosomal rearrangements, and the use of animals and cells obtained therefrom for therapeutic and experimental purposes. In particular, the invention encompasses methods for producing non-human mammals having monoclonal or oligoclonal peripheral B cell and T cell repertoires using mature B cells and T cells as donors for nuclear transfer. However, any donor cell containing chromosomal somatic rearrangements is a suitable donor cell in the present invention, particularly for methods involving the production of experimental animals.

For instance, olfactory and pheromone receptors expressed on the surface of sensory neurons undergo rearrangement and allelic exclusion in the generation of receptor diversity, and were recently likened to antigen receptors on the surface of immune cells. Boyd et al. 1998, J. Immunol. 161: 1718-27. Further, rearrangement of odorant receptor genes may also be RAG-dependent in that rag1 was recently shown to be expressed in zebrafish olfactory sensory neurons. See Jessen et al., 2001, Genesis 29(4): 156-62. Thus, cells other than lymphocytes that contain somatic chromosomal rearrangements are suitable donor cells for the methods of the present invention, and may be used to develop cloned experimental animals for the study of cell differentiation and development and molecular mechanisms of allelic exclusion.

Preferred donor cells to be used in the methods of the invention are mature B and T lymphocytes. For instance, the invention includes a method for producing a non-human animal and preferably a mammal with a monoclonal or oligoclonal peripheral B cell repertoire, comprising (a) identifying and isolating a non-human mature B cell of interest or a nucleus from a non-human mature B cell of interest; (b) introducing said mature B cell or the nucleus of said mature B cell into a non-human enucleated oocyte and preferably a mammalian enuclated oocyte (or another suitable recipient cell) of the same species as the mature B cell or B cell nucleus to form a nuclear transfer (NT) unit; (c) implanting said NT unit into the uterus of a surrogate mother of said species; and (d) permitting the NT unit to develop into a non-human mammal having a monoclonal or oligoclonal B cell repertoire. Advanced Cell Technology, Inc. (the assignee of this application) and other groups have developed methods for transferring the genetic information in the nucleus of a somatic or germ cell from a child or adult into an unfertilized egg cell, and culturing the resulting cell to divide and form a blastocyst embryo having the genotype of the somatic or germ nuclear donor cell. Methods for cloning by such methods are referred to as “somatic cell nuclear transfer” because somatic donor cells are commonly used. Such methods, including methods by which the embryos produced by somatic cell nuclear transfer are transferred into a non-human female mammal of the same species to develop to term, are described, for example, in U.S. Pat. Nos. 5,994,619, 6,235,969, and 6,252,133, the contents of which are incorporated herein by reference in their entirety.

In the context of T cells, the invention includes a method for producing a non-human mammal with a monoclonal or oligoclonal T cell receptor repertoire, comprising (a) identifying and isolating a non-human mature CD4+ or CD8+ T cell of interest or a nucleus from a non-human T cell of interest; (b) introducing said T cell of interest or the nucleus of said T cell of interest into a non-human mammalian enucleated oocyte (or another suitable recipient cell) of the same species as the T cell of interest or T cell nucleus of interest to form a nuclear transfer (NT) unit; (c) implanting said NT unit into the uterus of a surrogate mother of said species; and (d) permitting the NT unit to develop into a non-human mammal having a monoclonal or oligoclonal T cell receptor repertoire.

“Mature” in the context of a B cell means a differentiated B cell that expresses a desirable immunoglobulin from rearranged Ig heavy and light chain genes. Mature B cells expressing a desirable immunoglobulin may be isolated using well known methods. For instance, mature B cells may be isolated following in vivo or in vitro immunization. Various types of plaque assays can be used to screen B lymphocytes in the absence of hybridoma formation in order to identify and isolate B cells expressing Ig specific for a particular antigen. For instance, see U.S. Pat. No. 5,627,052 of Schrader, herein incorporated by reference.

“Mature” in the context of T cells means a differentiated double positive (CD4(+)CD8(+)) or single positive CD4(+) or CD8(+) T cell that expresses a TcR from rearranged TcR alpha and beta genes, or gamma and delta genes. Mature T cells of interest may also be identified and isolated using techniques that are well known in the art. For instance, U.S. Pat. No. 6,255,073 of Cai et al., herein incorporated by reference in its entirety, describes a synthetic antigen-presenting matrix having the requisite costimulatory assistance and at least the extracellular portion of a Class I MHC molecule capable of binding to a selected peptide operably linked to the support. The synthetic matrix can be an entire cell or cell membrane expressing the MHC and costimulatory molecules. This synthetic antigen presenting system may be used activate a population of T-cell lymphocytes against a peptide of interest when the peptide is bound to the extracellular portion of the MHC molecule.

Activated T cells recognizing an antigen of interest may be separated and/or enriched by a variety of means, including indirect binding of cells to specially coated surfaces, Ficoll-Hypaque gradient centrifugation (Pharmacia, Piscataway, N.J.), or affinity-based separation techniques directed at the presence of either the CD4 or CD8 receptor antigens. These affinity-based techniques include flow microfluorimetry, including fluorescence-activated cell sorting (FACS), cell adhesion, and like methods. (See, e.g., Scher and Mage, in Fundamental Immunology, W. E. Paul, ed., pp. 767-780, River Press, NY (1984).) Affinity methods may utilize anti-CD4 and anti-CD8 receptor antibodies as the source of affinity reagent. Alternatively, the natural ligand, or ligand analogs, of either the CD4 or CD8 receptors may be used as the affinity reagent. Various anti-T-cell, i.e., anti-CD4 and anti-CD8 monoclonal antibodies, for use in these methods are generally available from a variety of commercial sources, including the American Type Culture Collection (Rockville, Md.) and Pharmingen (San Diego, Calif.). Negative selection procedures may also be used to effect the removal of undesirable cells from the donor cell purification procedure, or a combination of both negative and positive selection procedures. See, e.g. Cai and Sprent, J. Exp. Med. 179: 2005-2015 (1994).

U.S. Pat. No. 5,635,363 of Altman et al., herein incorporated by reference in its entirety, discloses methods and compositions for labeling T cells according to the specificity of their antigen receptor. Specifically, a stable multimeric complex is prepared with major histocompatibility complex protein subunits having a substantially homogeneous bound peptide population, which is used to formed a stable structure with T cells recognizing the complex through their antigen receptor, thereby allowing for the labeling, identification and separation of specific T cells.

The methods of the present invention may be performed using other donor cells, for instance embryonic cells or ES cells or differentiated fetal and adult cells such as fibroblast cells, wherein a whole immunoglobulin gene or genes and/or a whole TcR gene or genes are “knocked in.” Monoclonality or oligoclonality in the cloned cells and animals of the invention may maintained by removing endogenous V-D-J exons and “knocking in” one gene or cDNA or set of genes such that only one immunoglobulin or TcR may be made. B and T cells may also be used. In this manner, immunization is not required to isolate donor cells producing antigen-specific antibodies, because genes or cDNAs encoding for antigen-specific receptors may be identified separately and “knocked in.” Preferred genes are genes encoding antibodies specific for VEGFR1 and 2, EGF receptor and other receptors involved in cancer.

The methods of the present invention may be performed with donor cells and recipient oocytes of any animal species, including but not limited to human and non-human primate cells, ungulate, canine, feline, lagomorph, rodent, avian, and fish cells. Primate cells with which the invention may be performed include but are not limited to cells of humans, chimpanzees, baboons, cynomolgus monkeys, and any other New or Old World monkeys. Ungulate cells with which the invention may be performed include but are not limited to cells of bovines, porcines, ovines, caprines, equines, buffalo and bison. Rabbits are an example of a lagomorph species with which the invention may be performed. Chickens (Gallus gallus) are an example of an avian species with which the invention may be performed. Rodent cells with which the invention may be performed include but are not limited to mouse, rat, guinea pig, hamster and gerbil cells. Mice are useful as experimental animal models given the extensive work that has already been performed in identifying many genes involved in immune cell differentiation. Work with transgenic mice expressing heterologous TcR and Ig transgenes demonstrates the feasibility and exemplifies the utility and the of the disclosed methods.

Wakayama and colleagues recently demonstrated nuclear transfer in mice starting with late-passage ES cells. See Wakayama et al., 1999, Dev. Biol. 96(26): 14984-89, and published PCT application WO 0145500, each of which is herein incorporated by reference. The method involves microsurgical isolation of a donor nucleus followed by piezo-electrically actuated microinjection into an enucleated, unfertilized metaphase 11 oocyte. The method has also been shown to work with adult somatic cells, specifically, with the nuclei of cumulus cells isolated from adult females, and short-term cultured cells derived from the tails of adult males. See Wakayama et al., 1998, Nature 394: 369-74, and Wakayama et al., 1999, Nat. Genet. 22: 127-28, each of which is incorporated in its entirety.

Murine B and T cells expressing a particular Ig gene or TcR gene of interest can be used as donor cells in practicing the invention. For example, a murine cell that produces a human immunoglobulin, i.e., a cell from a transgenic mouse expressing a rearranged human Ig or human TcR beta and/or alpha transgenes, can be used as a donor cell. Methods for engineering such mice are known in the art. For instance, Green and colleagues report “XenoMouse” strains of mice that are genetically engineered with megabasesize YACs carrying portions of the human IgH and IgKappa loci, including the majority of the variable repertoire, which produce a robust secondary immune response upon immunization with human antigens. Green et al., 1999, J. Immunol. Methods 231(1-2): 11-23, herein incorporated by reference. Monoclonal antibodies isolated from XenoMouse animals have been shown to have therapeutic potential both in vitro and in vivo, and appear to have the pharmacokinetics of normal human antibodies. Using B cells derived from XenoMouse strains as donors for nuclear transfer would result in cloned animals expressing a single type of human antibody, and would serve as a source for large scale production of the antibody without the need for hybridoma formation.

Similarly, Lonberg and Kay disclose transgenic non-human animals for producing heterologous antibodies, wherein transgenic human immunoglobulin genes are employed that are capable of undergoing isotype switching to generate heterologous antibodies of multiple isotypes. See U.S. Pat. No. 5,625,126, herein incorporated by reference. Such transgenic animals could be used as a preliminary reservoir to isolate different donor B cells expressing antibodies having the same variable region but different isotypes. Such B cells could then be used as donors for nuclear transfer, in order to produce cloned animals that produce antibodies with a single variable region of a single isotype.

It is also possible to isolate antigen specific human T cells and B cells for use as donors in nuclear transfer, i.e., for the production of human stem cells containing rearranged Ig or TcR genes. Such donor cells may be isolated directly from human patients undergoing an immune response, i.e., to a tumor or viral antigen. Alternatively, such donor cells may be isolated following immunization, i.e., by using animals engrafted with human immune cells for the isolation of antigen specific cells following immunization with human antigens. Such animals provide a convenient means for isolating antigen-specific human cells for therapy, given that humans cannot be immunized themselves with potentially harmful antigens for obvious ethical reasons.

For instance, in U.S. Pat. No. 5,698,767, herein incorporated by reference in its entirety, Wilson and Mosier demonstrate the transfer of immune cells from the human mononuclear phagocyte and lymphoid systems to a non-human laboratory animal of a different species. The nonhuman species to which the human immune cells are transferred can be any animal in which has a severely deficient immune system or lacks a functioning immune system, i.e. SCID mouse, SCID horse, etc. In SCID mice transplanted with adult human peripheral blood leukocytes (PBLs), the transplanted human PBLs were shown to expand in number and survive for at least fifteen months, and have been shown to reconstitute human immune function at both the T and B cell levels. Furthermore, specific human antibody responses were produced upon immunization.

Similarly, U.S. Pat. No. 5,849,288, which is also incorporated by reference in its entirety, discloses a method of producing animals with chimeric engrafted immune systems, wherein such animals are engrafted with xenogeneic hematopoietic cells and can produce xenogeneic, preferably human, B and/or T cells upon immunization with a suitable antigen. This patent purports to overcome some of the deficiencies that can be encountered when engrafting human T cells into SCID mice, including the observation that that some cells T cells succumb to functional anergy. The present invention should also overcome such limitations, in that T and B cells of interest will be rejuvenated through the process of nuclear transfer.

The methods of the invention may be used to produce animals having either monoclonal or oligoclonal B cell or T cell repertoires. As discussed above, while expression of transgenic Ig or TcR chains has been shown to suppress endogenous Ig and TcR gene rearrangement and expression, usually the suppression is not complete. This has recently been attributed to V(H) gene rearrangement in the case of the Ig heavy chain gene, which may be a normal way of generating immunoglobulin diversity. A similar mechanism dubbed receptor revision works to create diversity at the TcR beta locus, despite the arrangement and expression of the transgenic TcR. Thus, animals cloned from mature B and T cells may demonstrate the same secondary rearrangements seen in transgenic mice, thereby leading to some level of oligoclonality.

In some embodiments, oligoclonality may be desirable, i.e., for the study of receptor diversification mechanisms. A phenomenon called trans-switching may also be encountered in the case of immunoglobulin genes, whereby the rearranged Ig gene containing the variable region of interest undergoes switch recombination with another Ig constant gene switch sequence (RSS) thereby operably linking the desired variable region to another constant region. The possibility to generate such variants in a recombination-facilitating background provides the opportunity to isolate the antibodies of different isotypes containing the desired variable region. In the case of non-human cells expressing human immunoglobulins, it provides the opportunity to isolate chimeric antibodies containing non-human constant regions. Such chimeric antibodies may be desirable, for instance, in cases where non-human effector functions are desirable. Such effector functions may be desirable, for instance, for use in animal disease models, or for therapies where human effector function is preferably avoided. Such uses of trans-switching in transgenic mice are discussed in U.S. Pat. No. 5,625,126, which is herein incorporated by reference in its entirety.

If monoclonality is desired, there are many ways available in the art for inhibiting expression of the other Ig or TcR allele or secondary receptor revision. One embodiment concerns the use of donor cells that are deficient in secondary recombination at the Ig heavy gene or TcR beta gene locus. For example, cells may be chosen which are either Rag1- and/or Rag2-deficient. The feasibility of such an approach is supported by the studies with transgenic mice described above. For instance, Cascalho and colleagues reported the development of a quasi-monoclonal mouse containing one rearranged V(D)J segment at the heavy chain locus, with the other heavy chain allele being nonfunctional. Cascalho et al., 1996, Science 272(5268): 1585. While the primary repertoire of this mouse was monospecific, somatic hypermutation and secondary rearrangements were shown to change the specificity of 20% of the antigen receptors on B cells. But when Cascalho et al introduced homozygous, nonfunctional RAG-2 alleles into the quasi-monoclonal (QM) mouse, they found that the secondary repertoire was no longer diversified. See Cascalho et al., 1999, Dev. Immunol. 7(1): 43-50. Another group has also shown that simultaneous introduction of rearranged heavy chain and light chain transgenes into RAG2(−/−) mice leads to the generation of mice with monoclonal peripheral B cells. See Young et al., 1994, Genes Dev. 8(9): 1043-57. It is also possible to mate the cloned animals described herein with RAG knockout animals which already exist in the art in order to provide monoclonal animals according to the disclosed invention.

Thus, it is possible to take a donor B or T cell of interest, and genetically alter the cell so as to preclude further recombination at the immune receptor loci, i.e., by knocking out Rag1 and/or Rag2 expression. Alternatively, cloned mammals may be bred with a Rag deficient mammal to generate offspring that are Rag-deficient and monoallelic for either Ig or TcR expression. U.S. Pat. No. 5,583,278 of Alt et al., herein incorporated by reference in its entirety, discloses methods of making a recombinant mouse with both alleles of rag2 functionally deficient. Rag gene expression may be inhibited by either creating a homozygous deletion at either the rag1 or rag2 locus, or via antisense or RNA inhibition, i.e., via an interfering molecule expressed from a heterologous gene. Such techniques are known and should be familiar to those of skill in the art. Rag1 and rag2 homologues have been cloned in a variety of species, including humans (Bories et al., 1991, Blood 78(8): 2053-61). Indeed, when compared with other previously reported Rag1 sequences, the predicted amino acid translation (1073 aa) of Rag1 from rainbow trout displayed a minimum of 78% similarity for the complete sequence and 89% similarity in the conserved region (aa 417-1042), suggesting that rag genes could be readily identified on the basis of homology from any species for targeted deletion in the claimed methods.

There are other ways to specifically inhibit expression of the alternative Ig or TcR allele besides inhibiting Rag gene expression. Such alternative embodiments could be used where monoclonality is desired in the B compartment, for instance, but mature T cells expressing rearranged T cell receptors are also desired. Likewise, such embodiments would also be useful where monoclonality in the T cell compartment is desired, but expression and rearrangement of Ig genes is desired. Such methods include but are not limited to methods for inhibiting the expression of the alternative antibody or TcR gene, or methods for suppressing the activity of the expressed protein, i.e., with antiserum suppression.

For instance, partial or complete suppression of Ig chain expression can be produced by injecting cloned animals with antisera against one or more Ig chains (U.S. Pat. No. 5,625,126, which is incorporated herein by reference). Antisera are selected so as to react specifically with one or more Ig chains but to have minimal or no cross-reactivity with the Ig chains encoded by the rearranged genes of interest. Thus, administration of selected antisera will suppress new Ig chain expression but permits expression of the Ig chain(s) encoded by the rearranged genes of the cloned cell. In embodiments wherein cloned mice are designed that express a rearranged human antibody for instance, such antisera may be specific for mouse Ig while at the same time not capable of binding to human Ig. Suitable antibody sources for antibody comprise: (1) monoclonal antibodies, such as a monoclonal antibody that specifically binds to a murine μ, γ, kappa, or lambda chains but does not react with the human immunoglobulin chain(s) encoded by the heterologous human Ig gene of the invention; (2) mixtures of such monoclonal antibodies, so that the mixture binds with multiple epitopes on a single species of Ig chain, or with multiple types of Ig chains (e.g., murine μ and murine γ, or with multiple epitopes and multiple chains); (3) polyclonal antiserum or mixtures thereof, typically such antiserum/antisera is monospecific for binding to a single species of Ig chain (e.g., murine μ or γ, murine kappa, murine lambda) or to multiple species of Ig chains, and most preferably such antisera possesses negligible binding to human immunoglobulin chains encoded by a transgene of the invention; and/or (4) a mixture of polyclonal antiserum and monoclonal antibodies binding to a single or multiple species of Ig chains, and most preferably possessing negligible binding to the human immunoglobulin chains encoded by the rearranged gene of the cloned donor cell of the invention.

Cell separation and/or complement fixation can be employed to provide the enhancement of antibody-directed cell depletion of lymphocytes expressing endogenous (e.g., murine) immunoglobulin chains. In one embodiment, for example, antibodies are employed for ex vivo depletion of murine Ig-expressing explanted hematopoietic cells and/or B-lineage lymphocytes obtained from a cloned mouse harboring a rearranged human Ig gene or genes. Thus, hematopoietic cells and/or B-lineage lymphocytes are explanted from the cloned nonhuman animal harboring a rearranged human Ig gene or genes (i.e. harboring both a human heavy chain gene and a human light chain gene) and the explanted cells are incubated with an antibody (or antibodies) which (1) binds to a nonhuman, i.e., murine immunoglobulin and (2) lacks substantial binding to human immunoglobulin chains encoded by the rearranged gene(s). Such antibodies are referred to as “suppression antibodies.” The explanted cell population is selectively depleted of cells which bind to the suppression antibody(ies); such depletion can be accomplished by various methods, such as (1) physical separation to remove suppression antibody-bound cells from unbound cells (e.g., the suppression antibodies may be bound to a solid support or magnetic bead to immobilize and remove cells binding to the suppression antibody), (2) antibody-dependent cell killing of cells bound by the suppression antibody (e.g., by ADCC, by complement fixation, or by a toxin linked to the suppression antibody), and (3) clonal anergy induced by the suppression antibody, and the like.

Frequently, antibodies used for antibody suppression of endogenous Ig chain production will be capable of fixing complement. It is frequently preferable that such antibodies may be selected so as to react well with a convenient complement source for ex vivo/in vitro depletion, such as rabbit or guinea pig complement. For in vivo depletion, it is generally preferred that the suppressor antibodies possess effector functions in the nonhuman cloned animal species; thus, a suppression antibody comprising murine effector functions (e.g., ADCC and complement fixation) generally would be preferred for use in cloned mice.

In one variation, a suppression antibody that specifically binds to a predetermined endogenous immunoglobulin chain is used for ex vivo/in vitro depletion of lymphocytes expressing an endogenous immunoglobulin. A cellular explant (e.g., lymphocyte sample) from a cloned nonhuman animal harboring a human immunoglobulin rearranged heterologous gene is contacted with a suppression antibody and cells specifically binding to the suppression antibody are depleted (e.g., by immobilization, complement fixation, and the like), thus generating a cell subpopulation depleted in cells expressing endogenous (nonhuman) immunoglobulins (e.g., lymphocytes expressing murine Ig). The resultant depleted lymphocyte population (T cells, human Ig-positive B-cells, etc.) can be transferred into a immunocompatible (i.e., MHC-compatible) nonhuman animal of the same species and which is substantially incapable of producing endogenous antibody (e.g., SCID mice, RAG-1 or RAG2 knockout mice). The reconstituted animal (mouse) can then be immunized with an antigen (or reimmunized with an antigen used to immunize the donor animal from which the explant was obtained) to obtain high-affinity (affinity matured) antibodies and B-cells producing such antibodies. Such B-cells may be used to generate hybridomas by conventional cell fusion and screened. Antibody suppression can be used in combination with other endogenous Ig inactivation/suppression methods (e.g., J_{H} knockout, C_{H} knockout, D-region ablation, antisense suppression, compensated frameshift inactivation).

In other embodiments, it is desirable to effect complete inactivation of the alternative endogenous Ig loci so that hybrid immunoglobulin chains comprising a human variable region and a non-human (e.g., murine) constant region cannot be formed (e.g., by trans-switching between the transgene and endogenous Ig sequences). Knockout mice bearing endogenous heavy chain alleles with are functionally disrupted in the J_{H} region only frequently exhibit trans-switching, typically wherein a rearranged human variable region (VDJ) encoded by a transgene is expressed as a fusion protein linked to an endogenous murine constant region, although other trans-switched junctions are possible. To overcome this potential problem, it is generally desirable to completely inactivate the alternative heavy chain locus by any of various methods, including but not limited to the following: (1) functionally disrupting and/or deleting by homologous recombination at least one and preferably all of the other allele's or endogenous heavy chain constant region genes, (2) mutating at least one and preferably all of the other allele's or endogenous heavy chain constant region genes to encode a termination codon (or frameshift) to produce a truncated or frameshifted product (if trans-switched), and other methods and strategies apparent to those of skill in the art. Deletion of a substantial portion or all of the heavy chain constant region genes and/or D-region genes may be accomplished by various methods, including sequential deletion by homologous recombination targeting vectors. Similarly, functional disruption and/or deletion of at least one endogenous light chain locus (e.g., kappa) to ablate endogenous light chain constant region genes may also be done.

In cases where the donor cell for nuclear transfer contains a heterologous rearranged Ig gene, it is also possible to employ a frame-shifted transgene wherein the heterologous transgene comprises a frameshift in the J segment(s) and a compensating frameshift (i.e., to regenerate the original reading frame) in the initial region (i.e., amino-terminal coding portion) of one or more (preferably all) of the transgene constant region genes. Trans-switching to an endogenous IgH locus constant gene (which does not comprise a compensating frameshift) will result in a truncated or missense product that results in the trans-switched B cell being deleted or non-selected, thus suppressing the trans-switched phenotype.

Antisense suppression and antibody suppression may also be used to effect a substantially complete functional inactivation of the alternative or endogenous Ig gene product expression (e.g., murine heavy and light chain sequences) and/or trans-switched antibodies (e.g., human variable/murine constant chimeric antibodies). Various combinations of the inactivation and suppression strategies may be used to effect essentially total suppression of the alternative or endogenous (e.g., murine) Ig chain expression.

The cloned animals and stem cells isolated by the methods of the present invention find particular use in the fields of transplantation and xenotransplantation. As discussed above in the Background of Invention, the fact that embryos and embryonic stem cells may be generated using the nucleus from an adult differentiated cell has exciting implications for the fields of organ, cell and tissue transplantation. In particular, embryonic stem cells generated from the nucleus of a B cell or T cell taken from a patient in need of a bone marrow transplant could be made and induced or permitted to differentiate into hematopoietic cells having the patient's own histocompatibility profile. Accordingly, the problem of transplantation rejection and the dangers of immunosuppressive drugs could be precluded.

As discussed in U.S. Pat. No. 6,235,970, embryonic stem cells or cells isolated from the inner cell mass of nuclear transfer units (cultured ICM cells or CICM cells) may be induced to differentiate into a desired cell type according to known methods. For example, as disclosed therein, embryonic stem cells may be induced to differentiate into hematopoietic stem cells by culturing such cells in differentiation medium and under conditions that provide for cell differentiation. Medium and methods that result in the differentiation of CICM cells are known in the art as are suitable culturing conditions.

For example, Palacios and colleagues teach the production of hematopoietic stem cells from an embryonic cell line by subjecting cells to an induction procedure comprising initially culturing aggregates of such cells in a suspension culture medium lacking retinoic acid followed by culturing in the same medium containing retinoic acid, followed by transferal of cell aggregates to a substrate which provides for cell attachment. Palacios et al, 1995, Proc. Natl. Acad. Sci. USA, 92:7530-7537. Others have shown that the in vitro derivation of hematopoietic cells from mouse ES cells is enhanced by addition of stem cell factor (SCF), IL-3, IL-6, IL-11, GM, CSF, EPO, M-CSF, G-CSF, LIF. Keller et al, 1993, Mol. Cell Biol. 13:473-486; Kennedy et al, 1997, Nature 386(6624):488-493, 1997; Biesecker et al, 1993, Exp. Hematology, 21:774778. U.S. Pat. No. 6,280,718, which is incorporated herein by reference in its entirety, describes a method for inducing human ES cells to differentiate into hematopoietic cells. The method comprises culturing the ES cells with mammalian hematopoietic stromal cells; e.g., bone marrow or yolk sac cells, to induce the ES cells to differentiate into hematopoietic precursor cells, and then culturing the hematopoietic precursor cells in methylcellulose-containing medium to produce colonies of hematopoietic cells. Murine ES cells can also generate hematopoietic stem cells (thyl⁺, SCA-I⁺, c-kit receptor⁺, lineage restricted marker negative (B-220, Mac-1, TEN 119, JORO 75. for Blymphocyte, myeloid, erythroid, T-lymphocyte, respectively)) when cultured on a stromal cell line in the presence of IL-3, IL-6 and fetal liver stromal cell line cultured supernatant. See U.S. Pat. No. 6,245,566, herein incorporated by reference.

Hematopoietic stem cells may also be permitted or induced to differentiate further into a B or T cell lineage. For instance, Nourrit and colleagues describe the isolation of B cells from mutlipotent hematopoietic cells isolated from pre-liver embryos, and the differentiation of these B cells into Ig-secreting cells upon LPS stimulation. Nourrit et al., 1998, J. Immunol. 160: 4254-61. Benveniste and colleagues teach the in vitro directed differentiation of bone marrow precursors down a T cell lineage in medium supplemented with supernatant from a thymoma cell line. Benveniste et al, Cell. Immunol., 1990, 127(1): 92-104. In cases where the cloned cells or mammals are oligoclonal with respect to Ig or TcR, particular cells expressing the desired Ig or TcR can be identified and isolated using techniques known in the art. For instance, Ehlich and colleagues teach a gene amplification assay that permits the examination of rearranged Ig genes in single cells. Ehlich et al. 1994, Curr. Biol. 4(7): 573-83.

Thus, using known methods and culture medium, one skilled in the art may culture CICM cells and other pluripotent cells isolated from the animals or nuclear transfer units of the invention to obtain desired differentiated cell types, e.g., hematopoietic cells, B lymphocytes, T lymphocytes, etc. For human transplantation, preferably such cells are derived using a donor cell from the patient in need of a bone marrow or other immune cell transplant. However, it is also possible to use donor cells from other human subjects, i.e., in the case where the patient to be treated has a deficient B or T cell compartment. It is further possible to create cloned animals for therapeutic purposes, i.e., xenotransplantation, wherein such animals produce B or T lymphocytes containing a specifically rearranged Ig and/or TcR genes.

For xenotransplantation applications, it may be desirable to begin nuclear transfer using a donor mature B or T cell of interest that is genetically engineered via insertion of a heterologous gene and/or deletion or disruption of a native gene such that transplantation incompatibility is alleviated. For instance, donor cells may be engineered to express a MHC molecule of the patient to be treated, or may be engineered to delete endogenous MHC genes. Other methods for alleviating xenotransplant rejection may also be used. For instance, U.S. Pat. No. 6,296,846, herein incorporated by reference in its entirety, discloses methods for inducing xenograft tolerance in a mammal, by depleting the mammal of mature T cells, NK cells and anti-xenogeneic antibodies.

Where the cloned cells of the invention will be used to treat a cancer patient, a preferred CD4+ or CD8+ donor T cell is one that expresses a TCR that binds with specificity to a tumor antigen alone or in the context of MHC. Likewise, a preferred donor B cell would be one that expresses an Ig receptor specific for a tumor antigen, i.e. EGF receptor. Alternatively, for treating viral infections, donor CD4+ or CD8+ T cells may express a TcR that binds with specificity to a viral antigen, alone or in the context of MHC. Likewise, donor B cells may express an Ig molecule having specificity for a viral antigen, i.e., HIV.

The therapeutic utility of T cells expressing receptors specific for viral and tumor antigens has recently been demonstrated using a technique called TcR gene transfer. In one study, T cells were “redirected” by introducing the genes for a virus-specific TcR. T cells expressing the new TcR expanded upon viral infection of mice and efficiently homed to effector sites. Kessels et al., 2001, Nat. Immunol. 2(10): 900-01. Similarly, activated human peripheral blood lymphocytes transduced with retroviral vectors expressing melanoma-specific TcR receptor genes bound specifically to peptide/MHC complexes and showed specific antitumor reactivity as well as lymphokine production. Willemson et al., 2000, Gene Ther. 7(16): 1369-77. See also Clay et al., 1999, J. Immunol. 163: 507-13, and Calogero et al., 2000, Anticancer Res. 20(3A): 1793-9. Another group recently demonstrated the transfer of HIV specificity to primary human T lymphocytes by introducing specific TcR genes. Cooper et al., 2000, J. Virol. 74(17): 8207-12.

Thus, the transfer of specific TcR genes to the T cells of patients in need of anti-viral or anti-tumor therapy has been shown to result in target-specific T cells that elicit immune responses against the antigen of interest. This suggests that T cells isolated from the cloned stem cells and mammals of the invention will also find a similar utility. In fact, particularly for the monoclonal embodiments disclosed herein, the present invention overcomes some of the existing deficiencies with TcR gene transfer, in that the competition of transferred TcR genes with endogenous TcR chains for the components of the TcR complex tends to result in decreased expression of the transduced TcR on the cell surface following TcR gene transfer. See Cooper, id.

Moreover, particularly in the case of AIDS patients, the cloning process may overcome the lack of responsiveness generally seen in the CD8+ T cell compartment, given the rejuvenation of the donor cell via nuclear transfer. See Jerhouni et al., 1997, Thymus 24(4): 203-19, herein incorporated by reference, for a discussion of the reduced CTL response of the CD8+ T cells of AIDS patients. For instance, while the transfer of AIDS-specific TcR to CD8+ T cells from AIDS patients may not be successful due to the decreased response of the CD8+ T cell compartment, CD8+ T cells isolated from the cloned cells and mammals of the present invention would not suffer from the same deficiencies and could be used as a source of functional AIDS-specific cytotoxic cells.

Suitable tumor cells amenable to targeting by the cloned hematopoietic cells and lymphocytes of the present invention may be any tumor cells. Such cells include, but are not limited to, epithelial tumor cells, mesenchymal tumor cells, hematopoietic tumor cells, carcinoma cells, sarcoma cells, leukemic and lymphoma cells, breast cancer cells, ovarian cancer cells, pancreatic cancer cells, brain cancer cells, neuroblastoma cells, lung cancer cells, prostate or bladder cancer cells, etc. Suitable tumor antigens recognized by the Ig and TcR of the selected donor cells of the invention may be any tumor antigen associated with a cancer which is amenable to recognition by an Ig or TcR, including but not limited to receptors overexpressed on cancer cells, i.e., the EGF receptor, the Lewisy-related carbohydrate (found on epithelial carcinomas), the IL-2 receptor p55 subunit (expressed on leukemia and lymphoma cells), the erbB2/pI85 carcinoma-related proto-oncogene (overexpressed in breast cancer), gangliosides (e.g., GM2, GD2, and GD3), epithelial tumor mucin (i.e., MUC-1), carcinoembryonic antigen, ovarian carcinoma antigen MOv-18, squamous carcinoma antigen 17-1A, malignant melanoma antigen MAGE and other melanoma-associated immunodominant epitopes derived from melanoma-associated antigens such as MART-1/Melan A, gp 100/Pmel 17, tyrosinase, Mage 3, p15, TRP-1, and .beta.-catenin (Tsomides et al., International Immunol., 9:327-338), BRCA polypeptides or immunodominant fragments thereof, KS 1/4 pan-carcinoma antigen (Perez and Walker, 1990, J. Immunol. 142:3662-3667; Bumal, 1988, Hybridoma 7(4):407415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63) and prostate specific membrane antigen, to name just a few.

As mentioned above, non-human cloned mammals of the present invention may be used in methods of producing in large scale a monoclonal antibody of interest. Such methods may comprise, for instance, immunizing a cloned non-human mammal produced by the methods of the invention, i.e., by nuclear transfer of a mature B cell of interest, with an antigen recognized by the original B cell donor, and isolating said antibody of interest from the peripheral blood of said mammal. Alternatively, hematopoietic stem cells or B-lineage cells or pre-B cells or mature B cells may be isolated from the non-human cloned mammals of the invention, and antibody production may be directed either by in vitro immunization or other activation, i.e., LPS activation.

Accordingly, the present invention encompasses methods for the isolation of non-human hematopoietic stem cells that differentiate into B cells expressing a desired immunoglobulin, comprising (a) identifying and isolating a non-human mature B cell of interest or a nucleus from a non-human mature B cell of interest as described above; (b) introducing said mature B cell or the nucleus of said mature B cell into a non-human mammalian enucleated oocyte or other suitable recipient cell of the same species as the mature B cell or B cell nucleus to form a nuclear transfer (NT) unit; (c) implanting said NT unit into the uterus of a surrogate mother of said species; (d) permitting the NT unit to develop into a non-human fetus; and (e) isolating fetal liver hematopoietic stem cells (HSCs) from said non-human fetus, wherein said HSCs differentiate into B cells that express the desired immunoglobulin.

Alternatively, human or non-human hematopoietic stem cells that differentiate into B cells expressing a desired immunoglobulin may be isolated by a method comprising: (a) identifying and isolating a human or non-human mature B cell of interest or a nucleus from a human or non-human mature B cell of interest; (b) introducing said mature B cell or the nucleus of said mature B cell into a human or non-human enucleated oocyte or other suitable recipient cell; i.e., embryonic or hematopoietic stem cells, of the same species as the mature B cell or B cell nucleus to form a nuclear transfer (NT) unit; (c) activating the resultant NT unit; (d) culturing said activated NT unit until at least a size suitable for obtaining inner cell mass (ICM) cells; (e) disassociating said activated NT unit to obtain isolated ICM cells; (f) culturing said ICM cells obtained from said cultured NT unit to obtain embryonic stem cells; (g) permitting or directing said stem cells to develop into hematopoietic stem cells (HSCs) and isolating said HSCs, wherein said HSCs differentiate into B cells that express the desired immunoglobulin. Such HSCs could then be induced to differentiate into either B cells or T cells (depending which is the focus), any of which could be used in the therapeutic applications described herein.

The methods of the present invention can also be used to produce cloned cells and mammals expressing both monoclonal (or oligoclonal) B and T cell repertoires, i.e., by recloning using cells from cloned mammals as donor cells in subsequent rounds of nuclear transfer. Rag-deficient animals expressing monoclonal B or T cell repertoires, for instance, will not be able to rearrange the other receptor genes, given that rag genes are required for both Ig and TcR gene rearrangement and therefore both B and T cell maturation. However, transgenes encoding Ig or TcR genes of interest could be transfected into donor cells taken from cloned mammals in order to generate recloned mammals monoclonal for both the B cell and T cell repertoire. Such recloning methodology is disclosed in application Ser. No. 09/655,815, which is herein incorporated by reference in its entirety.

Alternatively, animals having either a monoclonal or oligoclonal B cell repertoire, for instance, could be mated with an animal having a monoclonal or oligoclonal T cell repertoire, for instance, to generate animals that have both B cell and T cell monoclonal or oligoclonal repertoires. Interbreeding animals having one rearranged Ig locus or TcR locus with a similar cloned animal will enable the isolation of a homogenous clone with two identical rearranged alleles that produces a monoclonal repertoire without having to inactivate Rag gene function. Such interbreeding will allow for the production of animals having a monoclonal B or T cell repertoire, which also is able to produce a full range of cells of the other lineage.

For business purposes, the invention also includes oocytes and sperm for producing animals according to the invention. For instance, oocytes may be isolated from female cloned animals having oligoclonal or monoclonal B cell repertoires, and used or sold as an agricultural product, for instance for the production of animals producing specific antibodies or T cells. Similarly, sperm from male cloned animals may be used or sold for the production of animals producing specific B cells or T cells. Oocytes and sperm of the invention could be used or sold together, for instance to produce animals with combined monoclonal or oligoclonal repertoires, i.e., by in vitro fertilization.

Other variations of the invention disclosed herein that do not depart from the spirit and scope of the invention are also encompassed.

What is claimed:

1. A method for producing a non-human animal with a monoclonal or oligoclonal peripheral B cell repertoire, comprising:
(a) identifying and isolating a non-human mature B cell or a nucleus from a non-human mature B cell;
(b) introducing said mature B cell or the nucleus of said mature B cell into a non-human mammalian enucleated oocyte of the same species as the mature B cell or B cell nucleus to form a nuclear transfer (NT) unit;
(c) implanting said NT unit into the uterus of a surrogate mother of said species; and
(d) permitting the NT unit to develop into a non-human mammal having a monoclonal or oligoclonal B cell repertoire.

2. The method of claim 1, wherein said non-human animal is selected from the group consisting of cows, sheep, pigs, goats, horses, mice, rabbits, rats, guinea pigs and avians.

3. The method of claim 2, wherein said mature B cell is genetically altered with at least one insertion, deletion or disruption that would inhibit the rearrangement of Ig genes in a maturing B cell.

4. The method of claim 3, wherein said non-human animal is a mammal.

5. The method of claim 4, wherein said genetic alteration results in a Rag1- and/or Rag2-deficient cell.

6. The method of claim 5, wherein said Rag1- and/or Rag2-deficient cell contains a RAG1 (−/−) and/or RAG2(−/−) knockout.

7. The method of claim 4, wherein said mature mammalian B cell is a mouse cell that produces a human immunoglobulin.

8. The method of claim 2, wherein the mature B cell is genetically engineered via insertion of a heterologous gene or deletion or disruption of a native gene that alleviates transplantation incompatibility.

9. The method of claim 1, wherein said mature B cell produces an immunoglobulin that binds with specificity to a tumor antigen.

10. The method of claim 1, wherein said mature B cell produces an immunoglobulin that binds with specificity to a viral antigen.

11. A non-human animal with a monoclonal or oligoclonal peripheral B cell repertoire produced by the method of claim 1.

12. A method of producing in large scale a monoclonal antibody or an oligoclonal antibody repertoire to an antigen, comprising immunizing the nonhuman animal of claim 11 with an antigen recognized by the original B cell of interest, and isolating an antibody that binds specifically to the antigen from the peripheral blood of said mammal.

13. A method of producing in large scale a monoclonal antibody, comprising obtaining pre-B cells or mature B cells from the non-human animal of claim 11, directing the production of antibody by such cells either by in vitro immunization or other activation, and isolating the antibody produced thereby.

14. A method of isolating non-human hematopoietic stem cells that differentiate into B cells expressing a desired immunoglobulin, comprising:
(a) identifying and isolating a non-human mature B cell or a nucleus from a non-human mature B cell;
(b) introducing said mature B cell or the nucleus of said mature B cell into a non-human enucleated oocyte of the same species as the mature B cell or B cell nucleus to form a nuclear transfer (NT) unit;
(c) implanting said NT unit into the uterus of a surrogate mother of said species;
(d) permitting the NT unit to develop into a non-human fetus; and
(e) isolating from said non-human fetus liver hematopoietic stem cells (HSCs) that differentiate into B cells that express the desired immunoglobulin.

15. A method of isolating human or non-human hematopoietic stem cells that differentiate into B cells expressing a desired immunoglobulin, comprising:
(a) identifying and isolating a human or non-human mature B cell or a nucleus from a human or non-human mature B cell;
(b) introducing said mature B cell or the nucleus of said mature B cell into a human or non-human enucleated oocyte of the same species as the mature B cell or B cell nucleus to form a nuclear transfer (NT) unit;
(c) activating the resultant NT unit;
(d) culturing said activated NT unit until at least a size suitable for obtaining inner cell mass (ICM) cells;
(e) dissociating said activated NT unit to obtain isolated ICM cells;
(f) culturing said ICM cells obtained from said cultured NT unit to obtain embryonic stem cells; and
(g) permitting or directing said stem cells to develop into hematopoietic stem cells (HSCs) and isolating said HSCs, wherein said HSCs differentiate into B cells that express the desired immunoglobulin.

16. A method for producing a non-human animal with a monoclonal or oligoclonal T cell receptor repertoire, comprising:
(a) enucleating an oocyte of a non-human animal;
(b) identifying and isolating a CD4+ or CD8+ T cell or a nucleus from a CD4+ or CD8+ T cell of the same species as the oocyte;
(c) introducing said T cell or the nucleus of said T cell into the oocyte to form a nuclear transfer (NT) unit;
(d) implanting said NT unit into the uterus of a surrogate mother of said species; and
(e) permitting the NT unit to develop into a non-human animal having a monoclonal or oligoclonal T cell receptor repertoire.

17. The method of claim 16, wherein said non-human animal is selected from the group consisting of cows, sheep, pigs, goats, horses, mice, rabbits, rats, guinea pigs and avians.

18. The method of claim 17, wherein said T cell of interest is genetically modified with at least one insertion, deletion or disruption that that would inhibit the rearrangement of Ig genes in a maturing T cell.

19. The method of claim 18, wherein said non-human animal is a mammal.

20. The method of claim 19, wherein said genetic alteration results in a Rag1- and/or Rag2-deficient cell.

21. The method of claim 20, wherein said Rag1- and/or Rag2-deficient cell contains a RAG1(−/−) and/or RAG2(−/−) knockout.

22. The method of claim 19, wherein the donor T cell is a mouse cell that produces a human TcR.

23. The method of claim 16, wherein the donor T cell is genetically engineered via insertion of a heterologous gene or deletion or disruption of a native gene that alleviates transplantation incompatibility.

24. The method of claim 16, wherein the donor T cell expresses a TCR that binds with specificity to a tumor antigen.

25. The method of claim 16, wherein the donor T cell produces a TCR that binds with specificity to a viral antigen.

26. A non-human animal with a monoclonal or oligoclonal peripheral T cell receptor repertoire produced by the method of claim 16.

27. The method of claim 1, wherein said non-human animal is a mammal.

28. A method of isolating non-human hematopoietic stem cells that differentiate into T cells expressing a desired TCR, comprising:
(a) enucleating an oocyte of a non-human mammal;
(b) identifying and isolating a CD4+ or CD8+ T cell or a nucleus from a CD4+ or CD8+ T cell of the same species as the oocyte;
(c) introducing said T cell or the nucleus of said T cell into the oocyte to form a nuclear transfer (NT) unit;
(d) implanting said NT unit into the uterus of a surrogate mother of said species;
(e) permitting the NT unit to develop into a non-human fetus; and
(f) isolating fetal liver hematopoietic stem cells (HSCs) from said non-human fetus, wherein said HSCs differentiate into T cells that express the desired TCR.

29. A method of isolating human or non-human hematopoietic stem cells that differentiate into T cells expressing a desired TCR, comprising:
(a) enucleating an oocyte of a human or a non-human mammal;
(b) identifying and isolating a CD4+ or CD8+ T cell or a nucleus from a CD4+ or CD8+ T cell of the same species as the oocyte;
(c) introducing said T cell or the nucleus of said T cell into the oocyte to form a nuclear transfer (NT) unit;
(d) activating the resultant NT unit;
(e) culturing the activated NT unit until at least a size suitable for obtaining inner cell mass (ICM) cells;
(f) disassociating the activated, cultured NT unit to obtain isolated ICM cells;
(g) culturing the isolated ICM cells to obtain pluripotent embryonic stem cells; and
(h) permitting or directing said pluripotent stem cells to develop into hematopoietic stem cells (HSCs), wherein said HSCs differentiate into T cells that express the desired TCR.

30. A method of treating cancer in an animal comprising transplanting the HSCs isolated by the method of claim 14 into said animal.

31. The method of claim 30, wherein said HSCs differentiate into monoclonal or oligoclonal B cells that express immunoglobulin specific for a receptor selected from the group consisting of VEGFR1, VEGFR2 and EGF receptor.

32. A method of treating cancer in an animal comprising transplanting the HSCs isolated by the method of claim 15 into said animal.

33. The method of claim 32, wherein said HSCs differentiate into monoclonal or oligoclonal B cells that express immunoglobulin specific for a receptor selected from the group consisting of VEGFR1, VEGFR2 and EGF receptor.

34. A method of treating cancer in an animal comprising transplanting the HSCs isolated by the method of claim 28 into said animal.

35. The method of claim 34, wherein said HSCs differentiate into monoclonal or oligoclonal T cells that express TcR specific for a receptor selected from the group consisting of VEGFR1, VEGFR2 and EGF receptor.

36. A method of treating cancer in an animal comprising transplanting the HSCs isolated by the method of claim 29 into said animal.

37. The method of claim 36, wherein said HSCs differentiate into monoclonal or oligoclonal T cells that express TcR specific for a receptor selected from the group consisting of VEGFR1, VEGFR2 and EGF receptor.
